(19) United States
(12) Patent Application Publication
(10) Pub. No.: US 2021/0340168 A1
(43) Pub. Date: Nov. 4, 2021

(54) OXA-SPIRODIPHOSPHINE LIGAND AND METHOD FOR ASYMMETRIC HYDROGENATION OF alpha, beta-UNSATURATED CARBOXYLIC ACIDS

(71) Applicant: SHENZHEN CATALYS TECHNOLOGY CO., LTD., Shenzhen (CN)

(72) Inventors: Genqiang CHEN, Shenzhen (CN); Jiaming HUANG, Shenzhen (CN); Yongpeng ZHENG, Shenzhen (CN); Baode MA, Shenzhen (CN); Xumu ZHANG, Shenzhen (CN)

(21) Appl. No.: 16/864,129

(22) Filed: Apr. 30, 2020

(51) Int. Cl. C07F 15/00; C07F 9/6561; C07C 227/16; C07D 211/22; C07D 405/12; B01J 31/24; B01J 31/22

(52) U.S. Cl. CPC C07F 15/0053; C07F 9/6561; C07C 227/16; C07D 211/22; B01J 2531/821; B01J 31/249; B01J 31/2213; B01J 2231/645; C07D 405/12

(57) ABSTRACT

The present invention provides an oxa-spirodiphosphine ligand having a structure of general Formula (I) below:

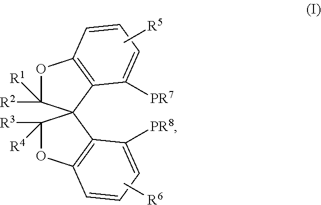

wherein in general Formula (I), R¹, R², R³ and R⁴ are the same, and are alkyl, alkoxy, aryl, aryloxy, or hydrogen, in which R¹, R², R³ and R⁴ may or may not form a ring, any two of them may form a ring, or a polycyclic ring may be formed between two pairs of them; R⁵ and R⁶ is alkyl, aryl, or hydrogen; and R⁷ and R⁸ is alkyl, benzyl, or aryl. The present invention also provides a method for asymmetric hydrogenation of α,β-unsaturated carboxylic acids. A complex of the oxa-spirodiphosphine ligand with ruthenium shows excellent activity and enantioselectivity in the asymmetric hydrogenation of various α,β-unsaturated carboxylic acids, with which a chiral carboxylic acid product can be obtained with an enantioselectivity up to 99%.

### TECHNICAL FIELD

The present invention relates to an oxa-spirodiphosphine ligand O-SDP and method for asymmetric hydrogenation of α, β-unsaturated carboxylic acids.

### BACKGROUND

Fragments of chiral carboxylic acids and chiral carboxylic acid derivatives are widely present in biologically active drug molecules and natural products, and have high potential for use in the field of asymmetric catalysis. The catalytic asymmetric hydrogenation of a, 0-unsaturated carboxylic acids is one of the most direct and effective ways to construct chiral carboxylic acid compounds. In the past few decades, with the development of chiral diphosphine ligands, this field has achieved rapid development. The ligands such as DIOP synthesized by Kagan's team (see Dang, T. P., Kagan, H. B. J. Chem. Soc. D: Chem. Commun. 1971, 481), BINAP developed by Noyori's team (see Miyashita, A.; Yasuda, A.; Takaya, H.; Toriumi, K.; Ito, T.; Souchi T.; Noyori R.; J. Am. Chem. Soc., 1980, 102, 7932; Kitamura, Masato; M. Tokunaga; and T. Ohkuma; R. Noyori. Org. Syn. 1998, 9,589), and DIPAM developed by Knowles' team (see Vineyard, B. D.; Knowles, W. S.; Sabacky, M. J.; Bachman, G. L.; and Weinkauff, D. J., J. Am. Chem. Soc. 1977, 99, 5946) are milestones in the development history of diphosphine ligands. They have been widely used in academia and industry. Then various chiral diphosphine ligands were synthesized, for example, SegePhos, DifluoPhos, SynPhos, Cₙ-TunePhos, TangPhos, DuanPhos, ZhangPhos, SKP, SDP, and SFDP. In 2007, Zhou Qilin's team found that the ruthenium complex of SFDP showed excellent activity and enantioselectivity in the hydrogenation of tiglic acid and α-methylcinnamic acid (see X. Cheng, Q. Zhang, J.-H. Xie, L.-X. Wang, Q.-L. Zhou, Angew. Chem., Int. Ed. 2005, 44, 1118-1121). ChenPhos and Trifer based on the ferrocene skeleton subsequently developed by Chen Weiping's group showed a good catalytic effect on the hydrogenation of α-methylcinnamic acid and α-oxo-α, β-unsaturated acids (see a) W. Chen, P. J. McCormack, K. Mohammed, W. Mbafor, S. M. Roberts, J. Whittall, Angew. Chem., Int. Ed. 2007, 46, 4141-4144; and b) W. Chen, F. Spindler, B. Pugin, U. Nettekoven, Angew. Chem., Int. Ed. 2013, 52, 8652-8656). Zhang Xumu's team also developed Wudaphos based on the ferrocene skeleton, which exhibited excellent activity and enantioselectivity in the hydrogenation of α-acrylic acid derivatives (see Chen, C.; Wang, H.; Zhang, Z., Jin, S.; Wen, S., Ji, J., Chung, L. W. Dong, X.-Q.; Zhang, X. Chem. Sci., 2016, 7, 6669-6673). However, the scope of application of these ligands is very narrow. In order to obtain excellent enantioselectivity, complex structural modifications of the ligands are often required, and there is still a lack of chiral diphosphine ligands that are widely applicable to various substrates. The Ir-SIPhOX system developed by Zhou Qilin's team is a catalytic system applicable to a wide range of substrates (see a) S. Li, S. F. Zhu, J. H. Xie, S. Song, C. M. Zhang, Q. L. Zhou, J. Am. Chem. Soc. 2010, 132, 1172-1179; b) S. Li, S. F. Zhu, C. M. Zhang, S. Song, Q. L. Zhou, J. Am. Chem. Soc. 2008, 130, 8584-8585; c) S. Song, S. F. Zhu, L. Y. Pu, Q. L. Zhou, Angew. Chem., It. Ed. 2013, 52, 6072-6075; d) S. Song, S. F. Zhu, Y. B. Yu, Q. L. Zhou, Angew. Chem., Int. Ed. 2013, 52, 1556-1559; e) Q. Wang, Z. Zhang, C. Chen, H. Yang, Z. Han, X.-Q. Dong, X. Zhang, Org. Chem. Front. 2017, 4, 627-630; and f) S. F. Zhu, Q. L. Zhou, Acc. Chem. Res. 2017, 50, 988-1001). The system is nonreactive for cyclic tetra-substituted carboxylic acids (see a) S. Song, S. F. Zhu, Y. Li, Q. L. Zhou, Org. Lett. 2013, 15, 3722-3725; and b) A. Schumacher, M. G. Schrems, A. Pfaltz, Chem.—Asian J. 2011, 17, 13502-13509), and needs an additional base to facilitate the progress of the reaction, which limits the scope of application of the system to some extent. Therefore, the development of new chiral ligands for the asymmetric hydrogenation of α, β-unsaturated carboxylic acids is of great significance.

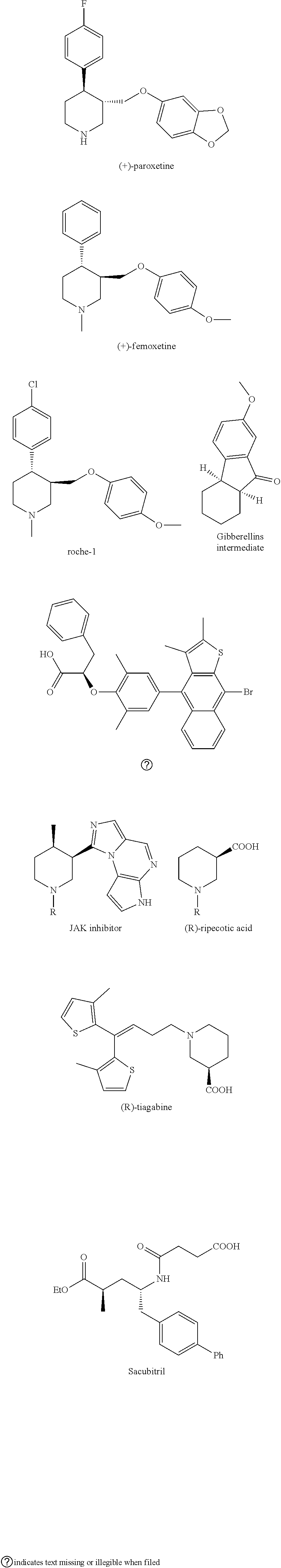

### SUMMARY

In view of the problems in the prior art that need to be overcome, the present invention provides an oxa-spirodiphosphine ligand. A complex of the ligand with ruthenium shows excellent activity and enantioselectivity in the asymmetric hydrogenation of various α,β-unsaturated carboxylic acids, with which a chiral carboxylic acid product can be obtained with an enantioselectivity up to 99%.

In order to achieve the above technical objectives, the following technical solutions are adopted in the present invention.

The present invention provides an oxa-spirodiphosphine ligand having a structure of general Formula (I) below:

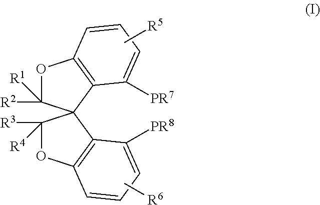

where in general Formula (I):
   R¹, R², R³ and R⁴ are the same, and are all alkyl, alkoxy, aryl, aryloxy, or hydrogen, where R¹,
   R², R³ and R⁴ may or may not form a ring, any two of them may form a ring, or a polycyclic ring may be formed between two pairs of them; R⁵, and R⁶ are alkyl, aryl, or hydrogen; and R⁷, and R⁸ are alkyl, benzyl or aryl.

In a further improved embodiment of the present invention, the oxa-spirodiphosphine ligand is the (±)-oxa-spirodiphosphine ligand, the (+)-oxa-spirodiphosphine ligand, or the (−)-oxa-spirodiphosphine ligand.

In a further improved embodiment of the present invention, the oxa-spirodiphosphine ligand comprises a compound having a structure below:

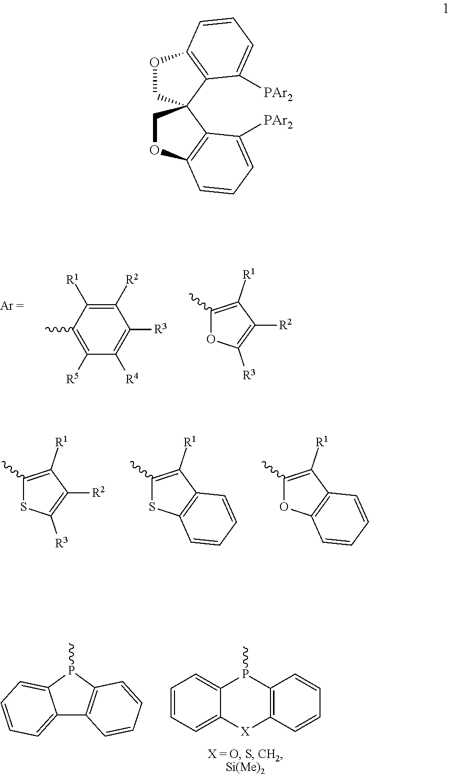

where R¹, R², R³, R⁴ and R⁵ are the same or different substituents, and include hydrogen, alkyl, fluoroalkyl, aryl, or alkoxy; and Ar is alkyl, benzyl or aryl.

In a further improved embodiment of the present invention, Ar is phenyl, or phenyl substituted with alkyl or alkoxy:

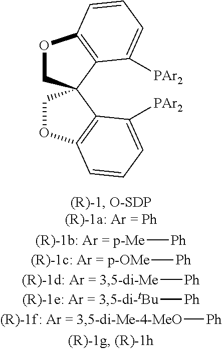

Another object of the present invention is to provide a method for asymmetric hydrogenation of α,β-unsaturated carboxylic acids, where the oxa-spirodiphosphine ligand is prepared into a diphosphine ruthenium acetate complex, with which asymmetric hydrogenation of α,β-unsaturated carboxylic acids is achieved in an organic solvent such as methanol, ethanol, trifluoroethanol, hexafluoroisopropanol, tetrahydrofuran, dioxane, toluene, benzene, methylene chloride, dichloroethane, methyl tert-butyl ether, diethyl ether or carbon tetrachloride.

In a further improved embodiment of the present invention, the diphosphine ruthenium acetate complex is a compound having a structure below:

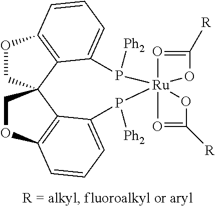

where R=alkyl, fluoroalkyl or aryl.

In a further improved embodiment of the present invention, the diphosphine ruthenium acetate complex is a product obtained by a synthesis route below:

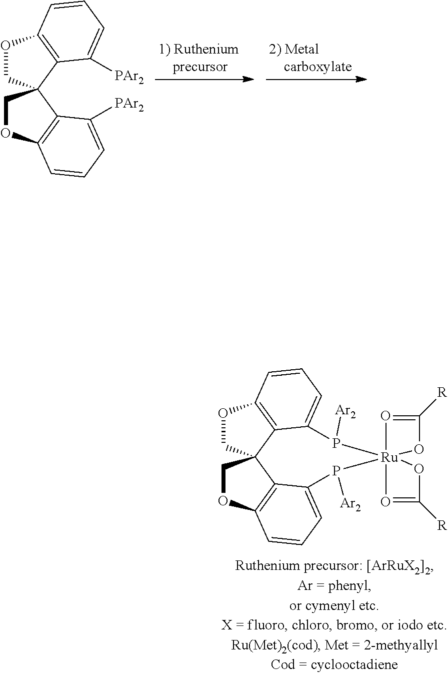

where R=alkyl, fluoroalkyl or aryl;
or is,

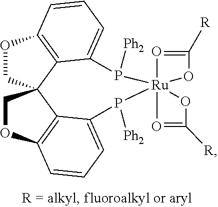

in which R=alkyl, fluoroalkyl or aryl.

In a further improved embodiment of the present invention, the diphosphine ruthenium acetate complex is used in the asymmetric reduction of a Sacubitril intermediate:

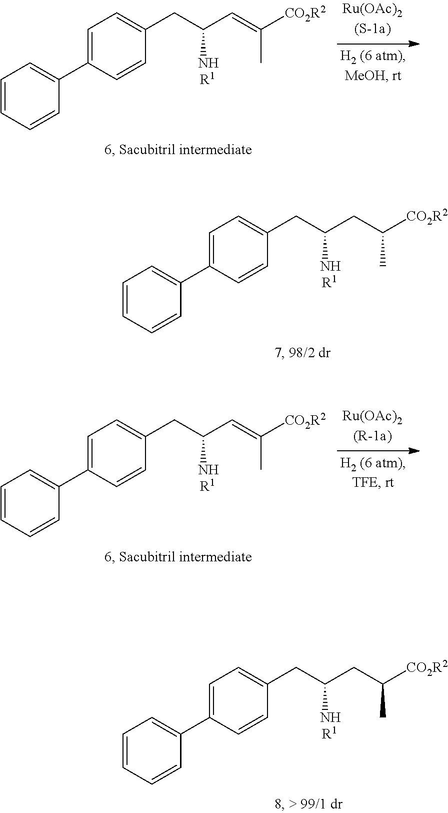

where R¹ is the Boc, Ts, N_{S}, Bn, PMB, PMP or Bz protecting group, R² is H, alkyl, or a substituted alkyl or aryl group. During the process, the ligand used is Compound 1

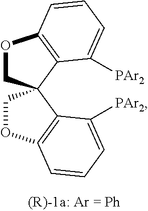

and more preferred structure is the ligand 1a

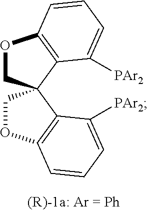

and the catalyst is used in an amount of substrate S/catalyst C=1 to 30000, further preferably substrate S/catalyst C=1000 to 15000, and further preferably substrate S/catalyst C=5000.

The solvent, temperature and hydrogen pressure used in this reaction are not limited to methanol, room temperature and 6 atmospheres. The solvent methanol is replaced by ethanol, or other organic solvents similar in nature to the above-mentioned organic solvents can be used. In addition to room temperature, the temperature can fluctuate in the range of room temperature±1-100 degrees Celsius, depending on the climate change. The pressure can also fluctuate in the range of 6 atmospheres±1 to 100 atm, depending on the climate change.

In a further improved embodiment of the present invention, the diphosphine ruthenium acetate complex is used in the asymmetric reduction of antidepressants paroxetine and femoxetine:

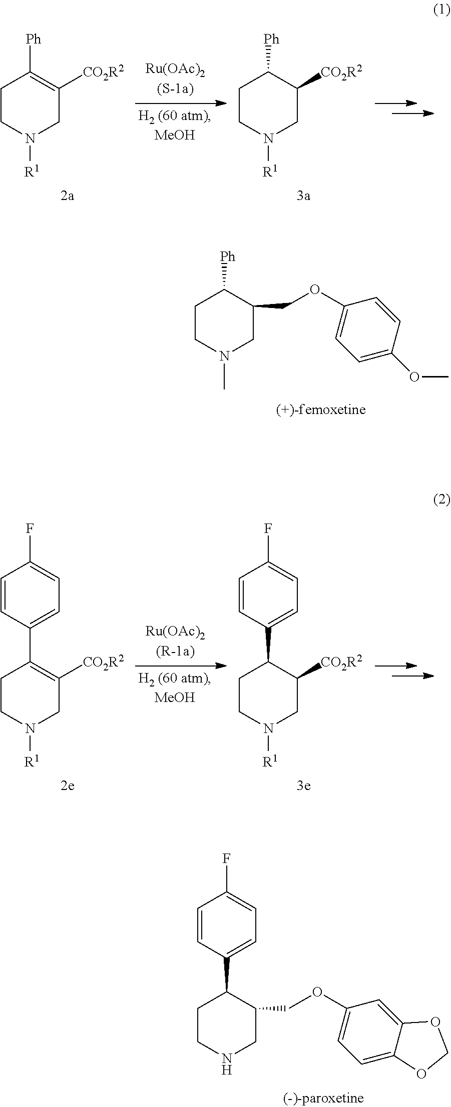

where R¹ is the Boc, Ts, N_{S}, Bn, PMB, PMP or Bz protecting group, R² is H, alkyl, or a substituted alkyl or aryl group. During the process, the ligand used is 1

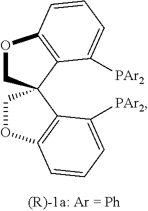

and more preferred structure is the ligand (R)-1a

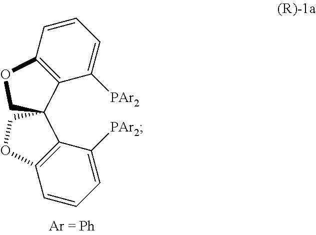

and the catalyst is used in an amount of substrate S/catalyst C=1 to 30000, further preferably substrate S/catalyst C=1000 to 15000, and further preferably substrate S/catalyst C=5000.

The solvent, temperature and hydrogen pressure used in this reaction are not limited to methanol, room temperature and 60 atmospheres. The solvent methanol is replaced by ethanol, or other organic solvents similar in nature to the above-mentioned organic solvents can be used.

In addition to room temperature, the temperature can fluctuate in the range of room temperature ±1-100 degrees Celsius, depending on the climate change. The pressure can also fluctuate in the range of 60 atmospheres±1 to 100 atm, depending on the climate change.

In a further improved embodiment of the present invention, the oxa-spirodiphosphine ligand is used as a catalyst in a reaction route shown below:

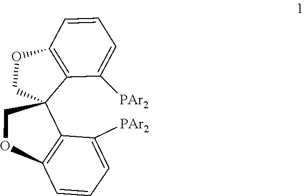

where R₁, R₂, and R₃=alkyl, fluoroalkyl or aryl, alkoxy and aryloxy; X is O, N, or S heteroatom, and n=0-10. The ligand used is 1

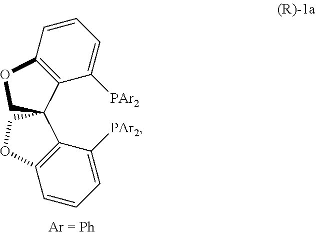

and more preferred structure is the ligand (R)-1a

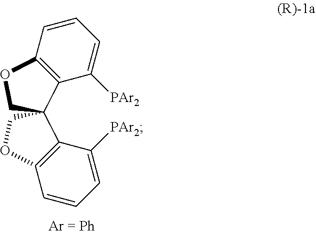

and the catalyst is used in an amount of substrate S/catalyst C=1 to 30000, further preferably substrate S/catalyst C=1000 to 15000, and further preferably substrate S/catalyst C=5000.

The solvent, temperature and hydrogen pressure used in this reaction are not limited to methanol, room temperature and 60 atmospheres. The solvent also includes, but is not limited to, ethanol, trifluoroethanol, hexafluoroisopropanol, tetrahydrofuran, dioxane, toluene, benzene, methylene chloride, dichloroethane, methyl tert-butyl ether, diethyl ether, carbon tetrachloride, and so on. In addition to room temperature, the temperature can fluctuate in the range of room temperature±1-100 degrees Celsius, depending on the climate change. The pressure can also fluctuate in the range of 60 atmospheres±1 to 100 atm, depending on the climate change.

Although O-SDP is similar in structure to SDP, it has a very unique structure where the angle of engagement is 99.2 degrees, which is greater than that of the known common chiral diphosphine ligands such as BINAP, MeO-BiPHEP, and Segphos, etc. A complex of the ligand with ruthenium shows excellent activity and enantioselectivity in the asymmetric hydrogenation of various α,β-unsaturated carboxylic acids, with which a chiral carboxylic acid product can be obtained with an enantioselectivity up to 99%. The synthesis method is applicable to the construction of core skeletons of chemical molecules with important biological activities such as Paroxetine, Femoxetine, nipecotic acid and Sacubitril.

The oxa-spirodiphosphine ligand O-SDP and the method for asymmetric hydrogenation of α,β-unsaturated carboxylic acids provided in the present invention have the following beneficial effects over the prior art:

(1) The oxa-spiro compound has central chirality, and thus include L-oxa-spirodiphosphine ligand and R-oxa-spirodiphosphine ligand. The racemic spirodiphosphine ligand can be synthesized from racemic oxa-spirobisphenol or oxa-spirodifluoride as a raw material.

(2) The present invention can be used as a chiral ligand in the asymmetric hydrogenation of unsaturated carboxylic acids, and its complex with ruthenium can achieve an enantioselectivity of greater than 99% in the asymmetric hydrogenation of methyl-cinnamic acid. That is, the diphosphine ruthenium acetate complex prepared from the compound has a high activity and an enantioselectivity of greater than 99% for the hydrogenation of a variety of unsaturated carboxylic acids in organic solvents.

### BRIEF DESCRIPTION OF THE DRAWINGS

FIG. 1 is a schematic view showing the present oxa-spirodiphosphine ligand O-SDP, preparation of various chiral compounds in the preparation of α,β-unsaturated carboxylic acids, and the corresponding conversion rates and enantioselectivity ee.

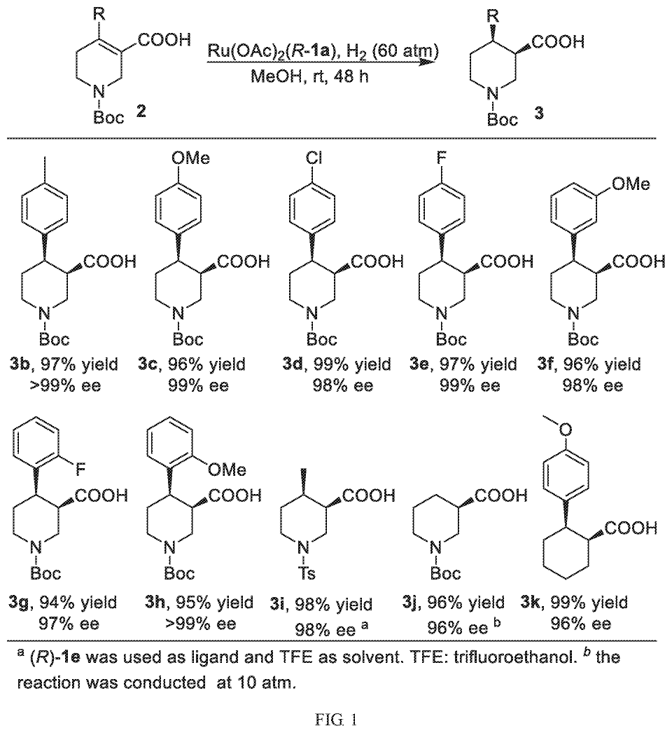

FIG. 2 is a schematic view showing the present oxa-spirodiphosphine ligand O-SDP, preparation of various chiral compounds in the preparation of α,β-unsaturated carboxylic acids, and the corresponding conversion rates and enantioselectivity ee.

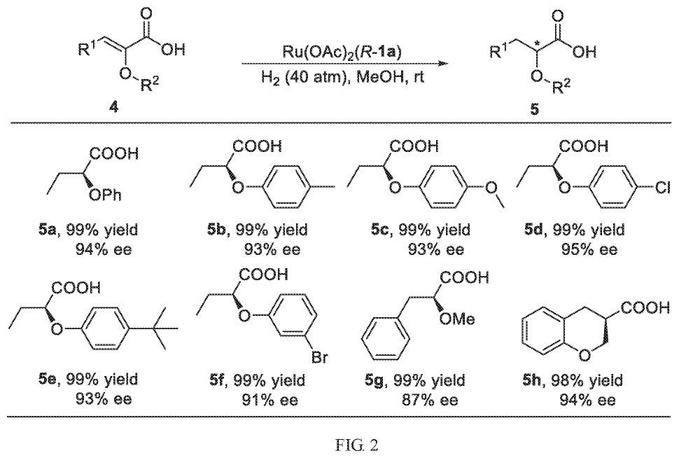

FIG. 3 is a schematic view showing the present oxa-spirodiphosphine ligand O-SDP, and the use of the chiral compounds prepared in the preparation of α,β-unsaturated carboxylic acids, in the synthesis of biologically active molecules.

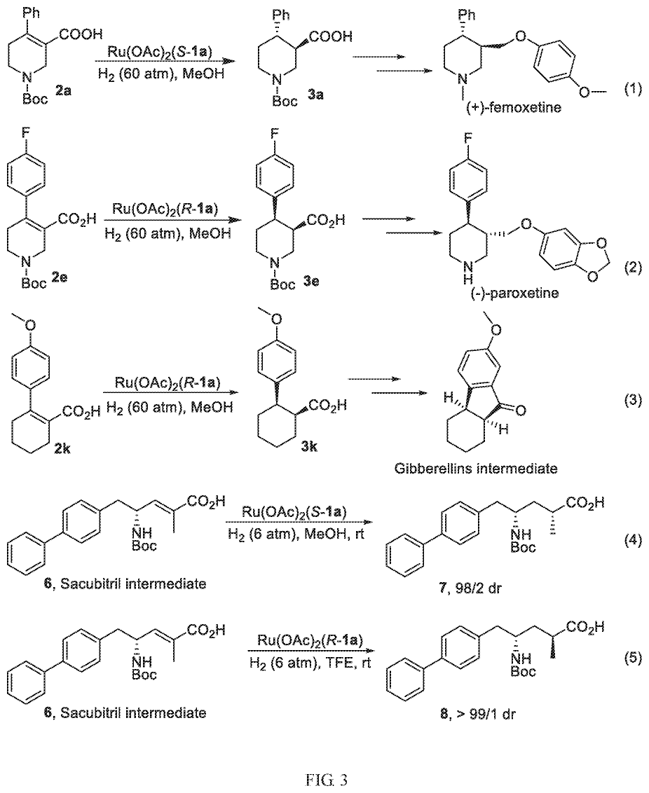

### DESCRIPTION OF THE EMBODIMENTS

The present invention will be described below by way of examples with reference to accompanying drawings. However, the oxa-spirodiphosphine ligand O-SDP and the method for asymmetric hydrogenation of α,β-unsaturated carboxylic acids provided in the present invention are not limited thereto.

In the field of chiral synthesis technology, although O-SDP is similar in structure to SDP, O-SDP has a very unique structure where the angle of engagement is 99.2 degrees, which is greater than that of the known common chiral diphosphine ligands such as BINAP, MeO-BiPHEP, and Segphos, etc. A complex of the ligand with ruthenium shows excellent activity and enantioselectivity in the asymmetric hydrogenation of various α,β-unsaturated carboxylic acids, with which a chiral carboxylic acid product can be obtained with an enantioselectivity up to 99%. The synthesis method by means of this route is applicable to the construction of core skeletons of chemical molecules with important biological activities such as Paroxetine, Femoxetine, nipecotic acid and Sacubitril.

The reaction principle is as follows.

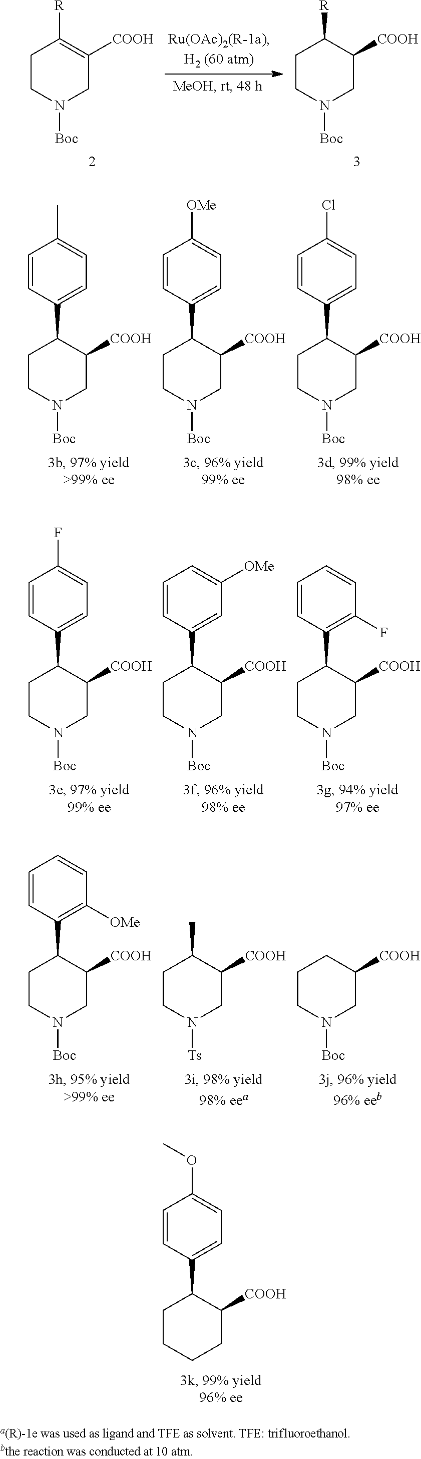

a (R)-1e is used as a ligand, TFE is used as a solvent, and the reaction time is 10 hrs.

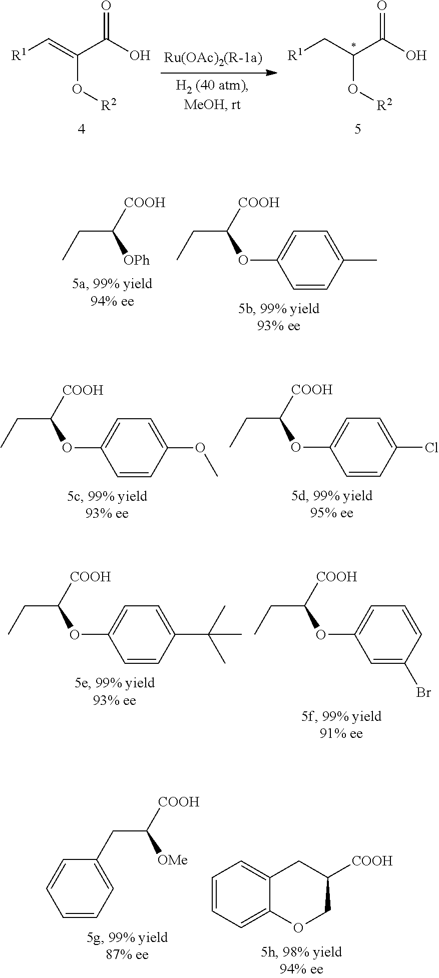

Yield represents the yield of the product.

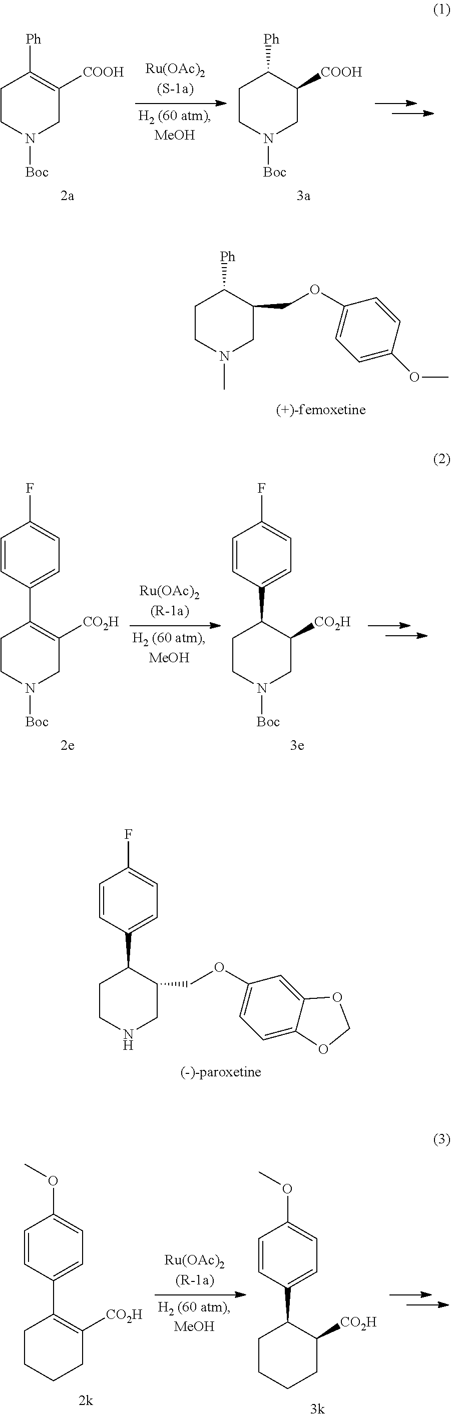

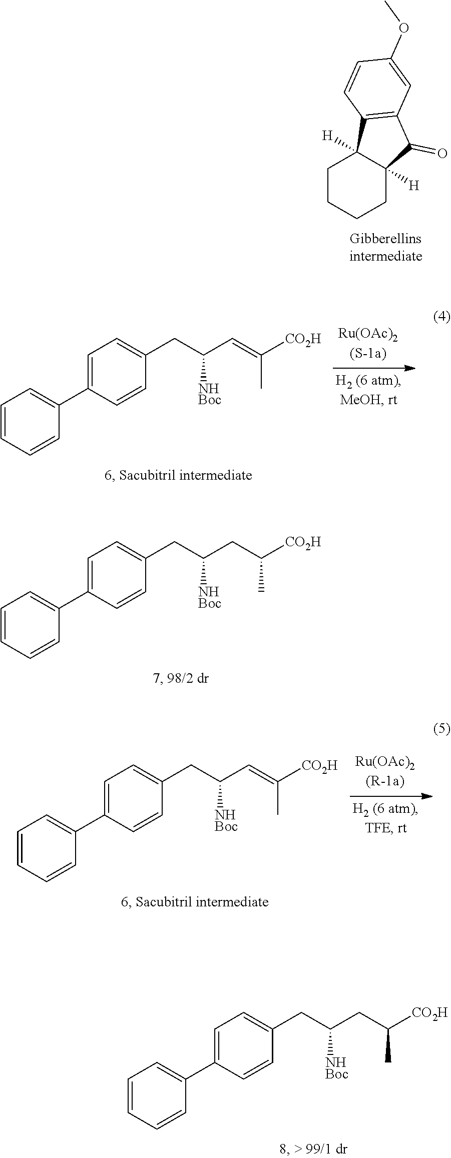 [SMILES: COc1ccc2c(c1)C(=O)[C@H]1CCCC[C@@H]21 ; C/C(=C\[C@@H](Cc1ccc(-c2ccccc2)cc1)NC(=O)OC(C)(C)C)C(=O)O ; C[C@H](C[C@@H](Cc1ccc(-c2ccccc2)cc1)NC(=O)OC(C)(C)C)C(=O)O ; C/C(=C\[C@@H](Cc1ccc(-c2ccccc2)cc1)NC(=O)OC(C)(C)C)C(=O)O ; C[C@@H](C[C@@H](Cc1ccc(-c2ccccc2)cc1)NC(=O)OC(C)(C)C)C(=O)O]

### Example 1

### Preparation of the Catalyst Ru(1a)OAc₂

Under a N₂ atmosphere, [RuPhCl₂]₂ (25 mg, 0.05 mmol) and the ligand 1a (61 mg, 0.103 mmol) were added to a 10 mL one-neck flask, and then DMF (2 mL) were added. The reaction was continued at 100° C. for 3 h. After cooling to room temperature, 1.5 mL of a solution of anhydrous sodium acetate (0.111 g, 1.3 mmol) in methanol was added. After 20 min, deoxygenated deionized water was added. A gray solid was precipitated from the reaction system, and filtered out. The solvent and water were removed under reduced pressure to obtain the catalyst Ru(1a)OAc₂ (57 mg, yield=71%).

### Example 2

### Preparation of Catalyst Ru(1a)(CF₃CO)₂

Under a N₂ atmosphere, bis(2-methylallyl)-cycloocta-1,5-diene ruthenium (32 mg, 0.05 mmol) and the ligand 1a (61 mg, 0.103 mmol) were added to a 10 mL one-neck flask, and then acetone (2 mL) were added. The reaction was continued at 40° C. for 0.5 h. Then, trifluoroacetic acid (33 mg, 0.3 mmol) was added and stirred overnight at 40° C. The solvent was removed under reduced pressure, and then petroleum ether (1 mL) was added, and filtered to obtain the target product Ru(1a)(CF₃CO)₂ (81 mg, yield=88%).

### Example 3

### Synthesis of (3R,4R)-1-(t-butoxycarbonyl)-4-phenyl-3-carboxylic acid 3a

Under a N₂ atmosphere, 2a (0.1 mmol), the catalyst Ru(1a)OAc₂ (0.8 mg, 0.001 mmol), and methanol (1 mL) were added to a hydrogenation flask. After 24 h under a hydrogen atmosphere of 60 atm, the raw material was completely converted into a product. 29.0 mg, yield of product=95%, >99% ee, [a]²⁵_{D}=+38.0 (c=0.5, CHCl₃), yellow oil. ¹H NMR (400 MHz, CDCl₃) δ 7.29-7.24 (m, 2H, Ar), 7.23-7.17 (m, 3H, Ar), 4.44 (d, J=12.7 Hz, 1H, CH₂), 4.26 (d, J=9.0 Hz, 1H, CH₂), 3.16 (d, J=11.1 Hz, 1H, CH), 3.01-2.82 (m, 3H, CH₂), 2.55 (dt, J=12.0, 8.6 Hz, 1H, CH), 1.68 (dd, J=13.0, 2.8 Hz, 1H, CH₂), 1.39 (s, 9H, CH₃). ¹³C NMR (101 MHz, CDCl₃) δ 176.9, 154.7, 142.1, 128.3, 127.4, 126.6, 79.8, 46.1, 45.2, 43.8, 43.0, 28.2, 25.6. HRMS (ESI) calcd. for C₁₇H₂₂NO₄ [M-H]⁻: 304.1554, Found: 304.1556. HPLC conditions: Daicel AD-3, volume of injection: 2 μL (c=1 mg/mL), Hexane/IPA=97/3, 1.0 mL/Min, 208 nm, t_{R} (major)=29.6 Min, t_{R} (minor)=31.4 Min.

### Example 4

### Synthesis of (3R,4R)-1-(t-butoxycarbonyl)-4-p-methylphenyl-3-carboxylic acid 3b

Under a N₂ atmosphere, 2b (0.1 mmol), the catalyst Ru(1a)OAc₂ (0.8 mg, 0.001 mmol), and methanol (1 mL) were added to a hydrogenation flask. After 24 h under a hydrogen atmosphere of 60 atm, the raw material was completely converted into a product. 31.0 mg, yield of product=97%, >99% ee, [a]²⁵_{D}=+47.1 (c=0.5, CHCl₃), yellow oil. ¹H NMR (400 MHz, CDCl₃) δ 7.09 (q, J=8.2 Hz, 4H, Ar), 4.42 (d, J=13.2 Hz, 1H, CH₂), 4.24 (d, J=10.5 Hz, 1H, CH₂), 3.15 (d, J=11.8 Hz, 1H, CH), 3.00-2.78 (m, 3H, CH₂), 2.61-2.42 (m, 1H, CH), 2.30 (s, 3H, CH₃), 1.67 (dd, J=13.1, 2.9 Hz, 1H, CH₂), 1.40 (s, 9H, CH₃). ¹³C NMR (101 MHz, CDCl₃) δ 177.0, 154.7, 139.1, 136.1, 129.0, 127.2, 79.8, 46.0, 45.3, 43.9, 42.6, 28.2, 25.8, 20.9. HRMS (ESI) calcd. for C₁₈H₂₄NO₄ [M-H]⁻: 318.1711, Found: 318.1713. HPLC conditions: Daicel AD-3, volume of injection: 2 μL (c=1 mg/mL), Hexane/IPA=97/3, 1.0 mL/Min, 220 nm, t_{R}(major)=18.9 Min, t_{R} (minor)=21.9 Min.

### Example 5

### Synthesis of (3R,4R)-1-(t-butoxycarbonyl)-4-p-methoxyphenyl-3-carboxylic acid 3c

Under a N₂ atmosphere, 2c (0.1 mmol), the catalyst Ru(1a)OAc₂ (0.8 mg, 0.001 mmol), and methanol (1 mL) were added to a hydrogenation flask. After 24 h under a hydrogen atmosphere of 60 atm, the raw material was completely converted into a product. 32.2 mg, yield of product=96%, 99% ee, [a]²⁵_{D}=+29.5 (c=0.5, CHCl₃), yellow solid. ¹H NMR (400 MHz, CDCl3) δ 7.14 (d, J=8.7 Hz, 2H, Ar), 6.84-6.78 (m, 2H, Ar), 4.41 (d, J=13.6 Hz, 1H, CH₂), 4.24 (d, J=10.7 Hz, 1H, CH₂), 3.77 (s, 3H, CH₃), 3.14 (d, J=11.4 Hz, 1H, CH), 2.97-2.79 (m, 3H, CH₂), 2.51 (qd, J=12.2, 3.8 Hz, 1H, CH), 1.66 (dd, J=13.1, 2.9 Hz, 1H, CH₂), 1.40 (s, 9H, CH₃). ¹³C NMR (101 MHz, CDCl3) δ 176.9, 158.2, 154.7, 134.2, 128.4, 113.7, 79.8, 55.1, 45.9, 45.4, 43.9, 42.2, 28.2, 25.9. HRMS (ESI) calcd. for C₁₈H₂₄NO₅ [M-H]⁻: 334.1660, Found: 334.1662. HPLC conditions: Daicel AS-3, volume of injection: 2 μL (c=1 mg/mL), Hexane/IPA=97/3, 1.0 mL/Min, 230 nm, t_{R} (major)=16.5 Min, t_{R} (minor)=19.2 Min.

### Example 6

### Synthesis of (3R,4R)-1-(t-butoxycarbonyl)-4-p-chlorophenyl-3-carboxylic acid 3d

Under a N₂ atmosphere, 2d (0.1 mmol), the catalyst Ru(1a)OAc₂ (0.8 mg, 0.001 mmol), and methanol (1 mL) were added to a hydrogenation flask. After 24 h under a hydrogen atmosphere of 60 atm, the raw material was completely converted into a product. 33.6 mg, yield of product=99%, 98% ee, [a]²⁵_{D}=+47.9 (c=0.5, CHCl₃), white solid. ¹H NMR (500 MHz, CDCl₃) δ 7.26 (d, J=8.3 Hz, 2H, Ar), 7.16 (d, J=8.3 Hz, 2H, Ar), 4.48 (d, J=12.0 Hz, 1H, CH₂), 4.29 (d, J=9.8 Hz, 1H, CH₂), 3.14 (d, J=11.6 Hz, 1H, CH), 2.93 (ddd, J=42.0, 23.0, 10.6 Hz, 3H, CH₂), 2.53 (dd, J=20.9, 11.7 Hz, 1H), 1.68 (d, J=11.0 Hz, 1H, CH₂), 1.40 (s, 9H, CH₃). ¹³C NMR (126 MHz, CDCl₃) δ 175.9, 154.7, 140.6, 132.5, 128.8, 128.5, 80.0, 46.1, 45.1, 43.8, 42.4, 28.3, 25.5. HRMS (ESI) calcd. for C₁₇H₂₁ClNO₄ [M-H]⁻: 338.1165, Found: 338.1169. HPLC conditions: Daicel AS-3, volume of injection: 2 μL (c=1 mg/mL), Hexane/IPA=97/3, 1.0 mL/Min, 208 nm, t_{R} (major)=9.0 Min, t_{R} (minor)=10.0 Min.

### Example 7

### Synthesis of (3R,4R)-1-(t-butoxycarbonyl)-4-p-fluorophenyl-3-carboxylic acid 3e

Under a N₂ atmosphere, 2e (0.1 mmol), the catalyst Ru(1a)OAc₂ (0.8 mg, 0.001 mmol), and methanol (1 mL) were added to a hydrogenation flask. After 24 h under a hydrogen atmosphere of 60 atm, the raw material was completely converted into a product. 31.4 mg, yield of product=97%, 99% ee, [a]²⁵_{D}=+35.2 (c=0.5, CHCl₃), yellow solid. 1H NMR (400 MHz, CDCl3) δ 7.18 (dd, J=8.4, 5.4 Hz, 2H, Ar), 6.96 (t, J=8.7 Hz, 2H, Ar), 4.45 (d, J=12.9 Hz, 1H, CH₂), 4.28 (d, J=9.3 Hz, 1H, CH₂), 3.14 (d, J=10.9 Hz, 1H, CH), 2.99-2.80 (m, 3H, CH₂), 2.53 (dd, J=20.9, 12.0 Hz, 1H, CH), 1.66 (dd, J=13.0, 2.5 Hz, 1H, CH₂), 1.39 (s, 9H, CH₃). ¹³C NMR (101 MHz, CDCl3) δ 176.8, 161.6 (d, J=243.5 Hz), 154.7, 137.8 (d, J=2.9 Hz), 128.9 (d, J=7.9 Hz), 115.1 (d, J=20.9 Hz), 79.9, 46.0, 45.4, 43.8, 42.3, 28.2, 25.7. ¹⁹F NMR (376 MHz, CDCl3) δ −116.3. HRMS (ESI) calcd. for C₁₇H₂₁FNO₄ [M-H]⁻: 322.1460, Found: 322.1464. HPLC conditions: Daicel AS-3, volume of injection: 2 μL (c=1 mg/mL), Hexane/IPA=98/2, 0.8 mL/Min, 208 nm, t_{R} (major)=15.0 Min, t_{R} (minor)=19.6 Min.

### Example 8

### Synthesis of (3R,4R)-1-(t-butoxycarbonyl)-4-m-methoxyphenyl-3-carboxylic acid 3f

Under a N₂ atmosphere, 2f (0.1 mmol), the catalyst Ru(1a)OAc₂ (0.8 mg, 0.001 mmol), and methanol (1 mL) were added to a hydrogenation flask. After 24 h under a hydrogen atmosphere of 60 atm, the raw material was completely converted into a product. 32.2 mg, yield of product=96%, 98% ee, [a]²⁵_{D}=+50.3 (c=0.5, CHCl3), yellow solid. 1H NMR (400 MHz, CDCl3) δ 7.18 (t, J=7.9 Hz, 1H, Ar), 6.84-6.72 (m, 3H, Ar), 4.43 (d, J=12.9 Hz, 1H, CH₂), 4.24 (d, J=11.1 Hz. 1H, CH₂), 3.74 (s, 3H, CH₃), 3.15 (d, J=11.0 Hz, 1H, CH₃), 3.00-2.82 (m, 3H, CH₂), 2.53 (dt, J=20.7, 10.2 Hz, 1H, CH), 1.68 (dd, J=12.9, 2.5 Hz, 1H, CH₂), 1.40 (s, 9H, CH₃). ¹³C NMR (101 MHz, CDCl₃) δ 176.6, 159.5, 154.7, 143.8, 129.3, 119.7, 113.3, 112.0, 79.8, 55.0, 46.0, 45.2, 44.0, 43.1, 28.2, 25.8. HRMS (ESI) calcd. for C₁₈H₂₄NO₅ [M-H]⁻: 334.1660, Found: 334.1664. HPLC conditions: Daicel OJ-3, volume of injection: 2 μL (c=1 mg/mL), Hexane/IPA=95/5, 1.0 mL/Min, 208 nm, t_{R} (major)=15.1 Min, t_{R} (minor)=17.9 Min.

### Example 9

### Synthesis of (3R,4R)-1-(t-butoxycarbonyl)-4-o-fluorophenyl-3-carboxylic acid 3g

Under a N₂ atmosphere, 2g (0.1 mmol), the catalyst Ru(1a)OAc₂ (0.8 mg, 0.001 mmol), and methanol (1 mL) were added to a hydrogenation flask. After 24 h under a hydrogen atmosphere of 60 atm, the raw material was completely converted into a product. 30.4 mg, yield of product=94%, 97% ee, [a]²⁵_{D}=+16.9 (c=0.5, CHCl3), yellow solid. ¹H NMR (400 MHz, CDCl₃) δ 7.29-7.23 (m, 1H, Ar), 7.19 (tdd, J=7.3, 5.4, 1.6 Hz, 1H, Ar), 7.08-6.97 (m, 2H, Ar), 4.50 (d, J=14.1 Hz, 1H, CH₂), 4.33 (d, J=11.3 Hz, 1H, CH₂), 3.29 (dt, J=12.7, 3.9 Hz, 1H, CH₂), 3.15 (d, J=12.9 Hz, 1H, CH), 2.96 (s, 1H, CH₂), 2.87 (t, J=11.8 Hz, 1H, CH₂), 2.62 (tt, J=12.6, 6.4 Hz, 1H, CH), 1.59 (dd, J=13.0, 2.6 Hz, 1H, CH₂), 1.41 (s, 9H, CH₃). ¹³C NMR (101 MHz, CDCl₃) δ 176.0, 160.7 (d, J=243.4 Hz), 154.7, 128.9 (d, J=13.7 Hz), 128.7 (d, J=3.7 Hz), 128.2 (d, J=8.6 Hz), 124.0 (d, J=3.6 Hz), 115.0 (d, J=22.4 Hz), 79.9, 46.3, 44.1, 43.3, 36.0, 36.0, 28.3, 24.7. ¹⁹F NMR (376 MHz, CDCl₃) δ −119.0. HRMS (ESI) calcd. for C₁₇H₂₁FNO₄ [M-H]⁻: 322.1460, Found: 322.1463. HPLC conditions: Daicel AD-3, volume of injection: 2 μL (c=1 mg/mL), Hexane/IPA=97/3, 1.0 mL/Min, 208 nm, t_{R} (major)=15.2 Min, t_{R} (minor)=20.8 Min.

### Example 10

### Synthesis of (3R,4R)-1-(t-butoxycarbonyl)-4-o-methoxyphenyl-3-carboxylic acid 3h

Under a N₂ atmosphere, 2h (0.1 mmol), the catalyst Ru(1a)OAc₂ (0.8 mg, 0.001 mmol), and methanol (1 mL) were added to a hydrogenation flask. After 24 h under a hydrogen atmosphere of 60 atm, the raw material was completely converted into a product. 31.8 mg, yield of product=95%, >99% ee,[a]²⁵_{D}=+84.8 (c=0.5, CHCl3), white solid. ¹H NMR (400 MHz, CDCl₃) δ 7.29-7.23 (m, 1H), 7.19 (tdd, J=7.3, 5.4, 1.6 Hz, 1H), 7.08-6.97 (m, 2H), 4.50 (d, J=14.1 Hz, 1H), 4.33 (d, J=11.3 Hz, 1H), 3.74 (s, 3H, CH₃), 3.29 (dt, J=12.7, 3.9 Hz, 1H), 3.15 (d, J=12.9 Hz, 1H), 2.96 (s, 1H), 2.87 (t, J=11.8 Hz, 1H), 2.62 (tt, J=12.6, 6.4 Hz, 1H), 1.59 (dd, J=13.0, 2.6 Hz, 1H), 1.41 (s, 9H). ¹³C NMR (101 MHz, CDCl₃) δ 176.0, 161.9, 159.5, 154.7, 129.0, 128.9, 128.7, 128.7, 128.2, 128.1, 124.0, 124.0, 115.1, 114.8, 79.9, 46.3, 44.1, 43.3, 36.0, 36.0, 28.3, 24.7. HRMS (ESI) calcd. for C₁₈H₂₄NO₅[M-H]⁻: 334.1660, Found: 334.1664. HPLC conditions: Daicel AD-3, volume of injection: 2 μL (c=1 mg/mL), Hexane/IPA=97/3, 1.0 mL/Min, 220 nm, t_{R} (major)=23.4 Min, t_{R} (minor)=24.9 Min.

### Example 11

### Synthesis of (3R,4R)-4-methyl-1-tosylpiperidin-3-carboxylic acid 3i

Under a N₂ atmosphere, 2i (0.1 mmol), the catalyst Ru(1a)OAc₂ (0.8 mg, 0.001 mmol), and methanol (1 mL) were added to a hydrogenation flask. After 24 h under a hydrogen atmosphere of 60 atm, the raw material was completely converted into a product. 29.1 mg, yield of product=98%, 98% ee, [a]²⁵_{D}=+23.3 (c=0.5, CHCl3), white solid. ¹H NMR (400 MHz, CDCl₃) δ 7.66 (d, J=8.2 Hz, 2H, Ar), 7.34 (d, J=8.0 Hz, 2H, Ar), 3.49 (dd, J=11.4, 2.3 Hz, 1H, CH₂), 3.35-3.26 (m, 1H, CH₂), 2.83 (ddd, J=15.5, 13.6, 6.9 Hz, 2H, CH), 2.72 (td, J=11.4, 3.0 Hz, 1H, CH), 2.44 (s, 3H, CH₃), 1.84 (qd, J=9.0, 4.3 Hz, 1H, CH₂), 1.74-1.65 (m, 1H, CH₂), 0.86 (d, J=7.1 Hz, 3H, CH₃). ¹³C NMR (101 MHz, CDCl₃) δ 177.2, 143.6, 133.1, 129.7, 127.6, 44.4, 43.2, 42.1, 30.4, 28.6, 21.5, 13.9. HRMS (ESI) calcd. for C₁₄H₁₈NO₄S [M-H]⁻: 296.0962, Found: 296.0963. HPLC conditions: Daicel OJ-3, volume of injection: 2 μL (c=1 mg/mL), Hexane/IPA=95/5, 1.0 mi/Min, 208 nm, t_{R} (major)=41.0 Min, t_{R} (minor)=43.9 Min.

### Example 12

### Synthesis of (3R)-1-(t-butoxycarbonyl)piperidin-3-carboxylic acid 3j

Under a N₂ atmosphere, 2j (0.1 mmol), the catalyst Ru(1a)OAc₂ (0.8 mg, 0.001 mmol), and methanol (1 mL) were added to a hydrogenation flask. After 24 h under a hydrogen atmosphere of 60 atm, the raw material was completely converted into a product. 22.0 mg, yield of product=96%, 96% ee, [a]²⁵_{D}=−43.2 (c=0.5, CHCl3), white solid. ¹H NMR (400 MHz, CDCl₃) δ 4.10 (s, 1H), 3.95-3.83 (m, 1H), 3.04 (s, 1H), 2.92-2.78 (m, 1H), 2.55-2.41 (m, 1H), 2.07 (dd, J=12.6, 3.6 Hz, 1H), 1.78-1.57 (m, 2H), 1.46 (s, 10H). ¹³C NMR (101 MHz, CDCl₃) δ 178.9, 154.7, 79.9, 45.4, 43.6, 41.1, 28.3, 27.1, 24.1. HPLC conditions: Daicel AD-3, volume of injection: 2 μL (c=1 mg/mL), Hexane/IPA=97/3, 1.0 mL/Min, 210 nm, t_{R} (major)=13.3 Min, t_{R} (minor)=13.9 Min.

### Example 13

### Synthesis of (1S,2R)-2-p-methoxyphenylcyclohexan-1-carboxylic acid 3k

Under a N₂ atmosphere, 2k (0.1 mmol), the catalyst Ru(1a)OAc₂ (0.8 mg, 0.001 mmol), and methanol (1 mL) were added to a hydrogenation flask. After 24 h under a hydrogen atmosphere of 60 atm, the raw material was completely converted into a product. 22.0 mg, yield of product=96%, 96% ee, [a]²⁵_{D}=−43.2 (c=0.5, CHCl₃), white solid. ¹H NMR (400 MHz, CDCl₃) δ 4.10 (s, 1H), 3.95-3.83 (m, 1H), 3.04 (s, 1H), 2.92-2.78 (m, 1H), 2.55-2.41 (m, 1H), 2.07 (dd, J=12.6, 3.6 Hz, 1H), 1.78-1.57 (m, 2H), 1.46 (s, 10H). ¹³C NMR (101 MHz, CDCl₃) δ 178.9, 154.7, 79.9, 45.4, 43.6, 41.1, 28.3, 27.1, 24.1. HPLC conditions: Daicel AD-3, volume of injection: 2 μL (c=1 mg/mL), Hexane/IPA=97/3, 1.0 mL/Min, 208 nm, t_{R} (major)=17.5 Min, t_{R} (minor)=19.6 Min.

### Example 14

### Synthesis of (S)-2-phenoxybutyric acid 5a

Under a N₂ atmosphere, 4a (0.1 mmol), the catalyst Ru(1a)OAc₂ (0.8 mg, 0.001 mmol), and methanol (1 mL) were added to a hydrogenation flask. After 24 h under a hydrogen atmosphere of 60 atm, the raw material was completely converted into a product. 22.0 mg, yield of product=96%, 96% ee, [a]²⁵_{D}=−43.2 (c=0.5, CHCl₃), white solid. ¹H NMR (400 MHz, CDCl₃) δ 4.10 (s, 1H), 3.95-3.83 (m, 1H), 3.04 (s, 1H), 2.92-2.78 (m, 1H), 2.55-2.41 (m, 1H), 2.07 (dd, J=12.6, 3.6 Hz, 1H), 1.78-1.57 (m, 2H), 1.46 (s, 10H). ¹³C NMR (101 MHz, CDCl₃) δ 178.9, 154.7, 79.9, 45.4, 43.6, 41.1, 28.3, 27.1, 24.1. HPLC conditions: Daicel AD-3, volume of injection: 2 μL (c=1 mg/mL), Hexane/IPA=97/3, 1.0 mL/Min, 220 nm, t_{R} (minor)=12.1 Min, t_{R} (major)=13.9 Min.

### Example 15

Hydrogenation of Sacubitril intermediate 6:

Under a N₂ atmosphere, 6 (1 mmol) and the catalyst Ru(1a)OAc₂ (0.16 mg, 0.0002 mmol) were added to two hydrogenation flasks respectively, and finally 5 mL of dichloroethane or trifluoroethanol was separately added. After 24 h under a hydrogen atmosphere, the raw material was completely converted into a product. The diastereomer ratio of Compound 7 is 98/2, and the diastereomer ratio of Compound 8 is >99/1, as determined by HPLC. HPLC conditions: Daicel AS-3, volume of injection: 2 μL (c=1 mg/mL), Hexane/IPA=92/8, 1.0 mL/Min, 220 nm, t_{R} (7)=8.253 Min, t_{R} (8)=10.281 Min.

It is to be understood that these examples are merely illustrative of the present invention and are not intended to limit the scope of the present invention. In addition, it should also be understood that after reading the disclosure of the present invention, various changes, modifications, and/or variations can be made to the present invention by those skilled in the art, and all these equivalents also fall within the scope of protection as defined by the appended claims of the present application.

It can be known from common technical knowledge that the present invention can be implemented by other embodiments without departing from the spirit or essential characteristics thereof. Therefore, the embodiments disclosed above are merely illustrative, and not exhaustive.

All changes within the scope of the present invention or within the equivalent scope to the present invention are included in the present invention.

What is claimed is:

1. An oxa-spirodiphosphine ligand, having a structure of general Formula (I) below: 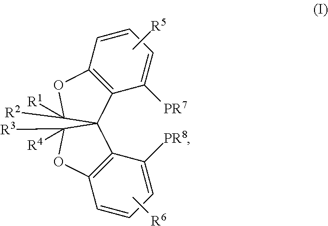
wherein in general Formula (I):
R¹, R², R³, and R⁴ are the same, and are all alkyl, alkoxy, aryl, aryloxy, or hydrogen, where R¹,
R², R³ and R⁴ may or may not form a ring, any two of them may form a ring, or a polycyclic ring may be formed between two pairs of them; R⁵ and R⁶ are alkyl, aryl, or hydrogen; and R⁷ and R⁸ are alkyl, benzyl, or aryl.

2. The oxa-spirodiphosphine ligand according to claim 1, which is the (±)-oxa-spirodiphosphine ligand, the (+)-oxa-spirodiphosphine ligand, or the (−)-oxa-spirodiphosphine ligand.

3. The oxa-spirodiphosphine ligand according to claim 1, comprising a compound having a structure below: 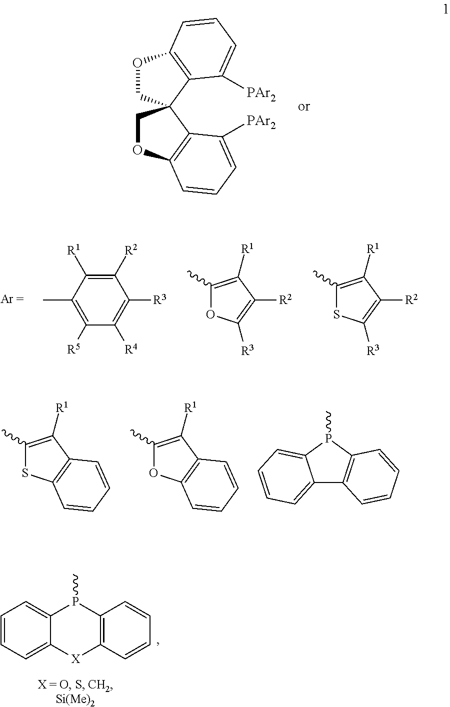 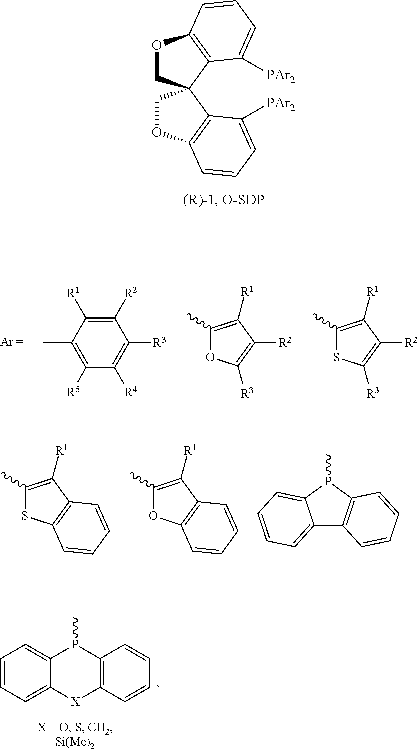
wherein R¹, R², R³, R⁴ and R⁵ are the same or different substituents, and include hydrogen, alkyl, fluoroalkyl, aryl, or alkoxy; and Ar is alkyl, benzyl or aryl.

4. The oxa-spirodiphosphine ligand according to claim 2, comprising a compound having a structure below: 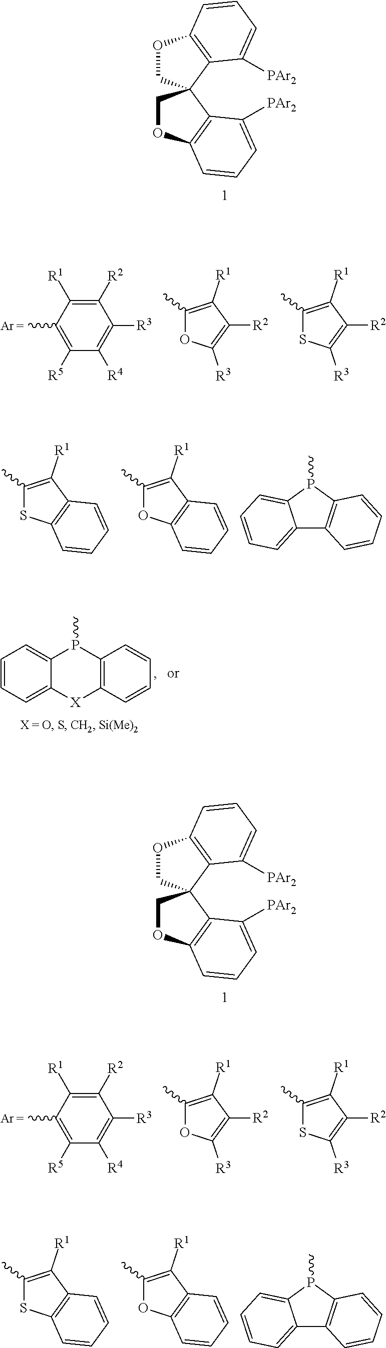 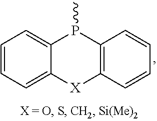
wherein R¹, R², R³, R⁴ and R⁵ are the same or different substituents, and include hydrogen, alkyl, fluoroalkyl, aryl, or alkoxy; and wherein Ar is alkyl, benzyl or aryl.

5. The oxa-spirodiphosphine ligand according to claim 3, wherein Ar is phenyl, or phenyl substituted with alkyl or alkoxy:

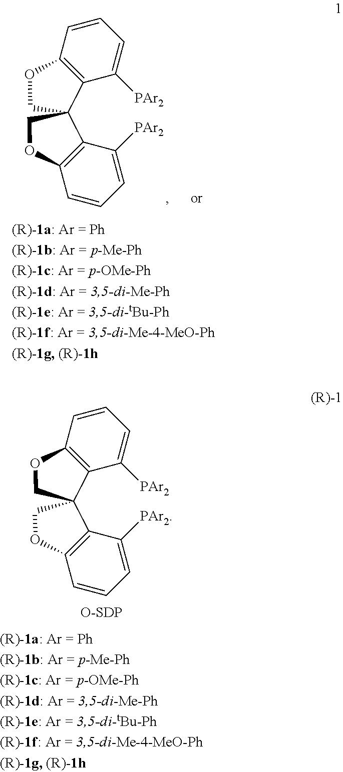

6. The oxa-spirodiphosphine ligand according to claim 4, wherein Ar is phenyl, or phenyl substituted with alkyl or alkoxy:

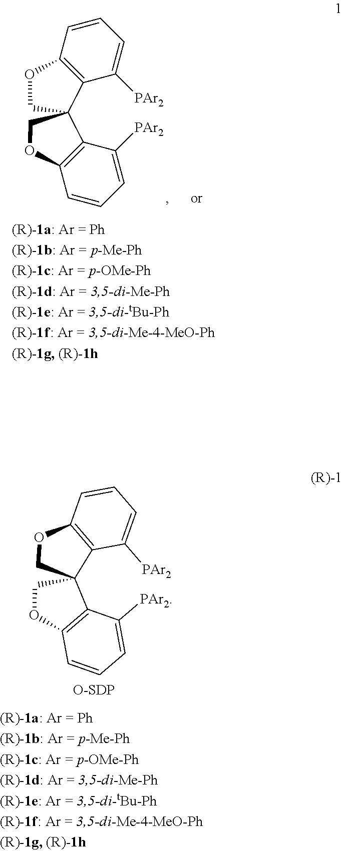

7. A method for asymmetric hydrogenation of α,β-unsaturated carboxylic acids, comprising the steps of:
preparing a diphosphine ruthenium acetate complex by using the oxa-spirodiphosphine ligand according to claim 1; and
achieving asymmetric hydrogenation of α,β-unsaturated carboxylic acids by placing the diphosphine ruthenium acetate complex in an organic solvent,
wherein the organic solvent is methanol, ethanol, trifluoroethanol, hexafluoroisopropanol, tetrahydrofuran, dioxane, toluene, benzene, methylene chloride, dichloroethane, methyl tert-butyl ether, diethyl ether or carbon tetrachloride.

8. The method according to claim 7, wherein the diphosphine ruthenium acetate complex is a compound having a structure below: 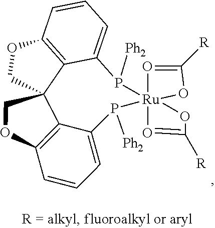
wherein R=alkyl, fluoroalkyl or aryl.

9. The method according to claim 7, wherein the diphosphine ruthenium acetate complex comprises a product obtained by a synthesis route below: 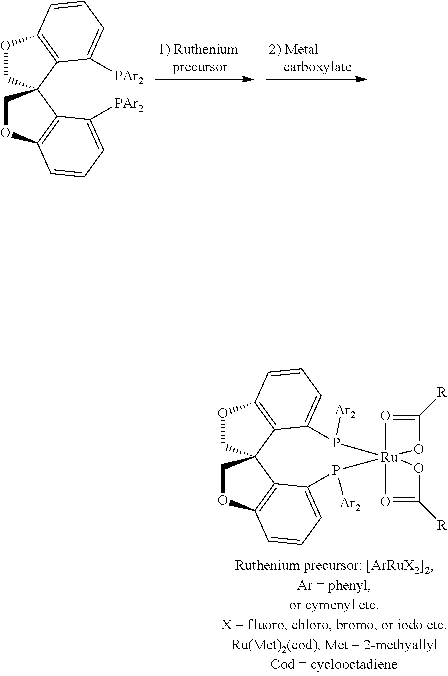
wherein R=alkyl, fluoroalkyl or aryl;
or is 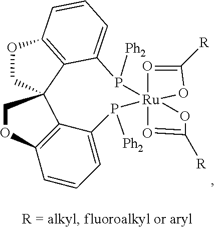
in which R=alkyl, fluoroalkyl or aryl.

10. The method according to claim 7, wherein the diphosphine ruthenium acetate complex is used in the asymmetric reduction of a Sacubitril intermediate: 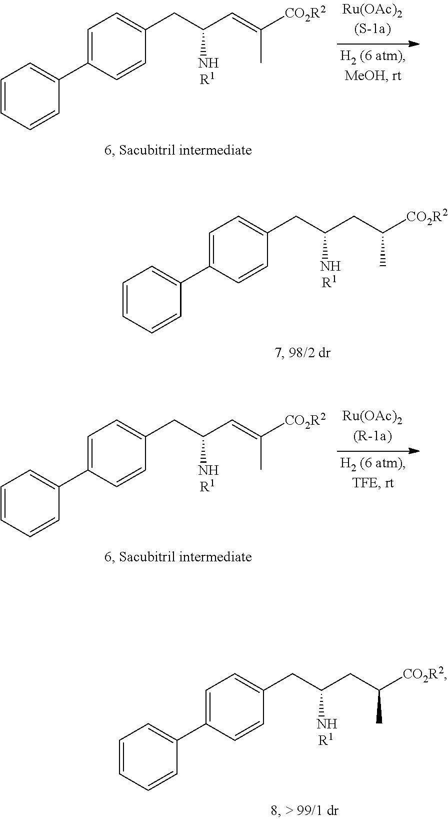
where R¹ is the Boc, Ts, N_{S}, Bn, PMB, PMP or Bz protecting group, R² is H, alkyl, or a substituted alkyl or aryl group, in which the ligand used is Compound 1 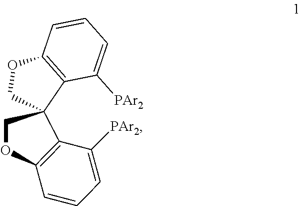
and wherein the catalyst is used in an amount of substrate S/catalyst C=1 to 30000.

11. The method according to claim 7, wherein the diphosphine ruthenium acetate complex is used in the asymmetric reduction of a Sacubitril intermediate: 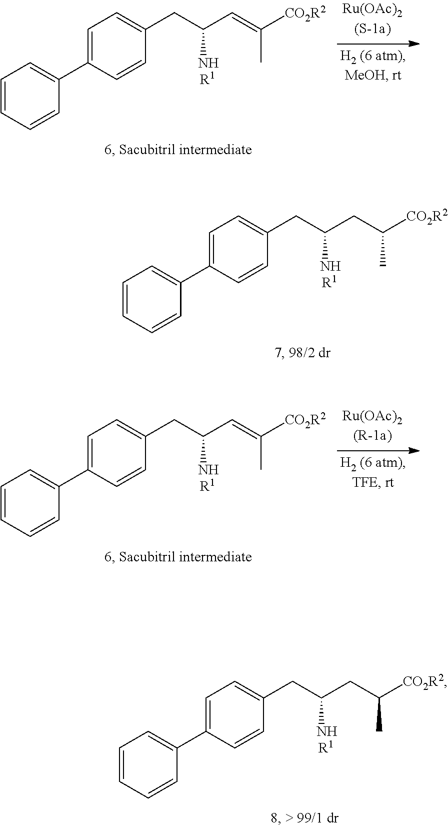
where R¹ is the Boc, Ts, N_{S}, Bn, PMB, PMP or Bz protecting group, R² is H, alkyl, or a substituted alkyl or aryl group, in which the ligand used is Compound 1a 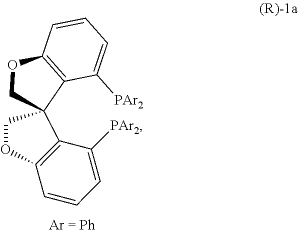
and wherein the catalyst is used in an amount of substrate S/catalyst C=1 to 30000.

12. The method according to claim 7, wherein the diphosphine ruthenium acetate complex is used in the asymmetric reduction of the intermediates of the antidepressants Paroxetine and Femoxetine: 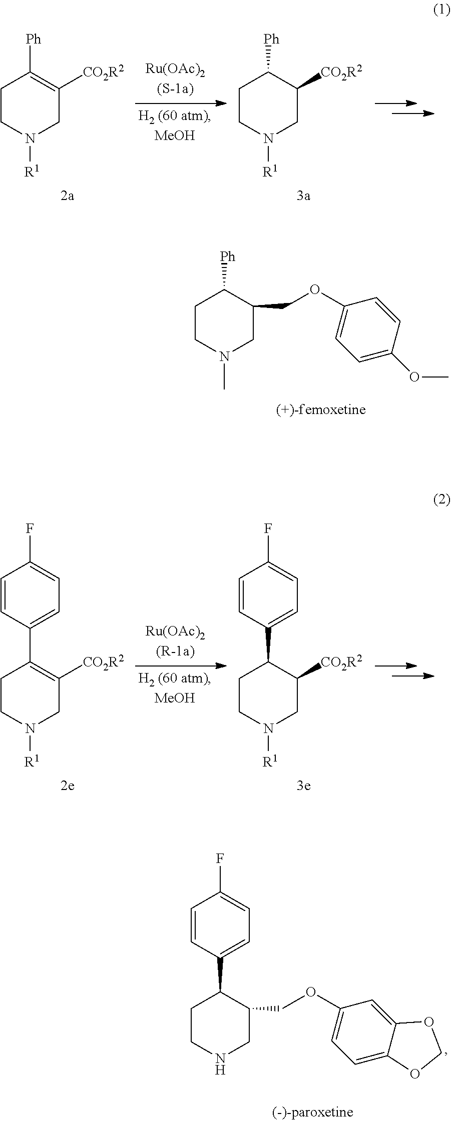
where R¹ is the Boc, Ts, N_{S}, Bn, PMB, PMP or Bz protecting group, R² is H, alkyl, or a substituted alkyl or aryl group, in which the ligand used is 1 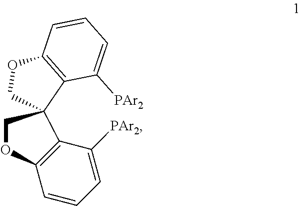
and wherein the catalyst is used in an amount of substrate S/catalyst C=1 to 30000.

13. The method according to claim 7, wherein the diphosphine ruthenium acetate complex is used in the asymmetric reduction of the intermediates of the antidepressants Paroxetine and Femoxetine: 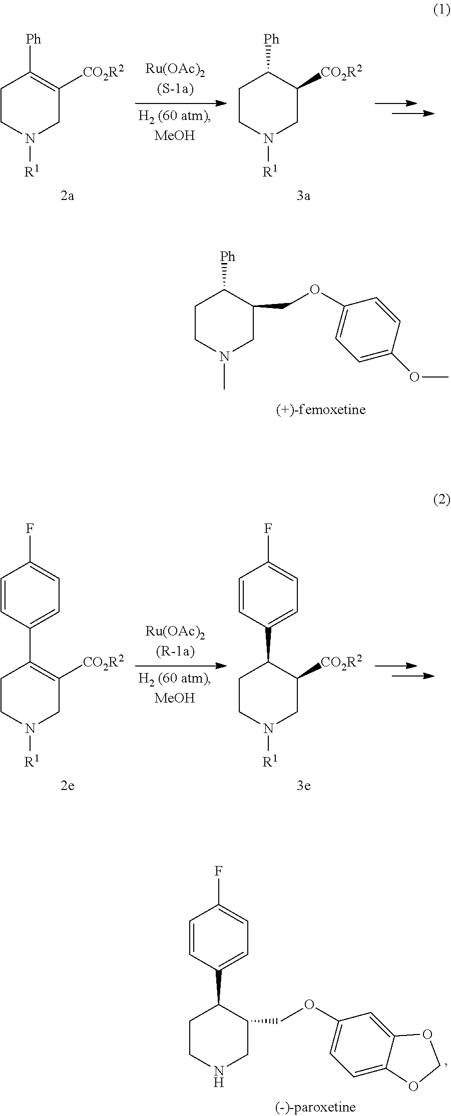
where R¹ is the Boc, Ts, N_{S}, Bn, PMB, PMP or Bz protecting group, R² is H, alkyl, or a substituted alkyl or aryl group, in which the ligand used is (R)-1a: 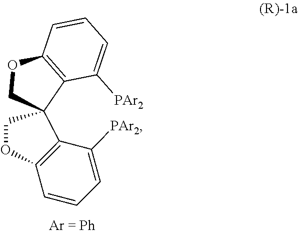
and wherein the catalyst is used in an amount of substrate S/catalyst C=1 to 30000.

14. The method according to claim 7, wherein the oxa-spirodiphosphine ligand is used as a catalyst in a reaction route shown below: 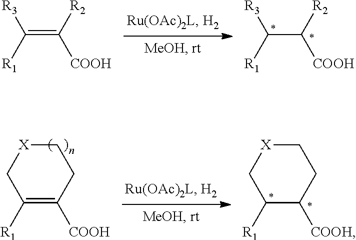
where R₁, R₂ and R₃=alkyl, fluoroalkyl or aryl, alkoxy, and aryloxy; X is O, N, or S heteroatom, and n=0-10, in which the ligand used is 1 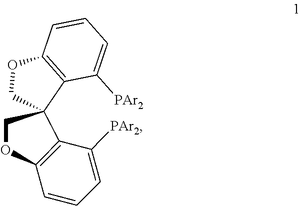
and wherein the catalyst is used in an amount of substrate S/catalyst C=1 to 30000.

15. The method according to claim 7, wherein the oxa-spirodiphosphine ligand is used as a catalyst in a reaction route shown below: 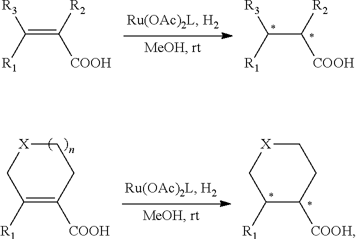
where R₁, R₂ and R₃=alkyl, fluoroalkyl or aryl, alkoxy, and aryloxy; X is O, N, or S heteroatom, and n=0-10, in which the ligand used is (R)-1a: 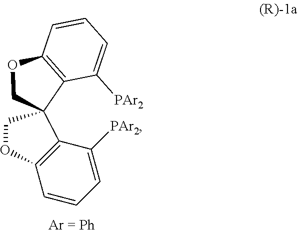
and wherein the catalyst is used in an amount of substrate S/catalyst C=1 to 30000.
